(19) United States
(12) Patent Application Publication
(10) Pub. No.: US 2023/0134725 A1
(43) Pub. Date: May 4, 2023

(54) Fusion Protein Extensions

(71) Applicants: Nant Holdings IP, LLC, Culver City, CA (US); NantBio, Inc., Culver City, CA (US)

(72) Inventors: Shahrooz Rabizadeh, Culver City, CA (US); Patrick Soon-Shiong, Culver City, CA (US); Kayvan Niazi, Culver City, CA (US)

(21) Appl. No.: 16/961,026

(22) PCT Filed: Jan. 16, 2019
(86) PCT No.: PCT/US19/13776

§ 371 Date: Oct. 15, 2020

Related U.S. Application Data
(60) Provisional application No. 62/630,182, filed on Feb. 13, 2018.
(60) Provisional application No. 62/618,639, filed on Jan. 18, 2018.

(51) Int. Cl. C07K 14/705; C07K 14/525; C07K 14/54; A61K 47/64

(52) U.S. Cl. CPC C07K 14/70532; C07K 14/70596; C07K 14/525; C07K 14/5443; A61K 47/643; A61K 47/64; C07K 2319/30; C07K 2317/24; C07K 2317/622; A61K 38/00

(57) ABSTRACT

Chimeric molecules and molecule complexes that include an Fc portion of an antibody and an immunomodulatory portion and uses thereof are contemplated. Typically, the chimeric molecule comprises at least two immune effectors, for example, IL-15 antagonist and PD-L1 binding domain. It is contemplated that a tumor cell or an immune competent cell can be exposed to the chimeric molecule to increase the effectiveness tumor cell lysis via an antibody-dependent cell-mediated cytotoxicity.

This application claims priority to US provisional applications with the Ser. No. 62/618,639, filed Jan. 18, 2018, and the Ser. No. 62/630,182, filed Feb. 13, 2018, both of which are incorporated by reference in their entireties herein.

### FIELD OF THE INVENTION

The field of the invention is recombinant proteins, and especially chimeric proteins that have immune modulatory functions.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Functional and effective T-cell responses are critical for a therapeutic immune response against infectious diseases and cancer. However, in certain circumstances such as chronic viral infections or cancer, the activity/response of various immune competent cells can be suppressed by certain immunosuppressive mechanisms exerted by the compromised cells. For example, T cells may lose their ability to proliferate, to produce effector molecules, and to lyse target cells. More recently, it was discovered that programmed death ligand-1 (PD-L1)/programmed death-1 (PD-1) interaction significantly contributes to the lack of T-cell responsiveness in settings of persistent antigenic stimulation, such as those encountered in cancer and chronic infectious diseases. Consequently, interference of the PD-1/PD-L1 interaction (e.g., using antibodies or targeted silencing) was thought to be a promising therapeutic approach to enhance cancer immunotherapy. In this context, it should be appreciated that PD-L1 is up-regulated by many cancer cells.

For example, inhibition of the negative co-stimulatory PD-1/PD-L1 pathway was targeted by transiently expressing the soluble extracellular part of PD-1 (sPD-1) or PD-L1 (sPD-L1) in human monocyte-derived DCs by mRNA electroporation as described in Gene Therapy (2014) 21, 262-271. In such approaches, multifunctional T cells were induced without induction of Treg cells. Presumably, relatively high concentrations of PD-1 and/or PD-L1 afforded sufficient interference with the PD-1/PD-L1 pathway to so induce a positive T cell response. Similarly, as described in U.S. Pat. No. 8,574,872, concatemeric PD-L1 extracellular domains were taught as a therapeutic agent for treatment of cancer. More specifically, the '872 patent discloses that the multimer comprising the extracellular domains of PD-1 or PD-L1 possess high antagonistic activity to their ligands, PD-L1 or PD-1, respectively, stimulate proliferation of lymphoid cells, and enhance their cellular cytotoxicity. While such soluble forms and multimers tend to reduce the chance of adverse immune responses that may be caused by traditional antibodies targeting PD-1 and PD-L1, serum half-life will likely be significantly reduced. Moreover, such soluble forms and multimers will be difficult to isolate from a recombinant source.

Further known therapeutic molecules include those in which Surfactant Protein D (SP-D) was used as a scaffold for multiple copies of CD40L and other TNF superfamily ligands (e.g., 4-1BBL, OX40L, GITRL) as described, for example, in U.S. Pat. No. 7,300,774. However, such constructs are generally difficult to produce and purify, and may be antigenic. In still other approaches using immune stimulatory molecules, single copies of OX40L, CD80, 4-1BBL, and GITRL were fused to an Fc portion of human immunoglobulin and used as adjuvant with cancer lysates (see Clin Cancer Res. 2012 Sep. 1; 18(17): 4657-4668; or U.S. Pat. No. 8,268,788, or U.S. Pat. No. 8,207,130). However, the biological activity of such constructs is often only apparent when used as adjuvants.

Thus, there is still a need for improved compositions and methods to treat cancer, and especially cancers that use PD-1/PD-L1 based immune evasion.

### SUMMARY OF THE INVENTION

The inventive subject matter is directed to various immune modulatory chimeric protein compositions and methods for immune therapy of cancer in which multimeric immune effectors are coupled to or present in a fusion protein that includes an Fc portion of an antibody.

In one aspect of the inventive subject matter, the inventors contemplate a chimeric molecule that comprises an Fc portion of an antibody and an immunomodulatory portion, wherein the immunomodulatory portion comprises at least two immune effectors. Most typically, the Fc portion comprises a portion of an IgG, while the immune effector comprises at least a portion (and most typically an extracellular portion) of PD-L1, a PD-L1 binder, PD-1, a PD-1 binder, PD-L2, a PD-L2 binder, 4-1BB, OX40L, and/or GITRL 4-1BB, OX40L, and/or GITRL. In some embodiments, the immune effectors are in a concatemeric arrangement, and in other embodiments the immune effectors are present at two distinct portions of the chimeric molecule.

It is still further contemplated that the chimeric molecule may also include a cytokine receptor, or a cytokine or cytokine analog bound to the cytokine receptor. In such case, one or more of the immune effectors may be covalently bound the cytokine receptor, the cytokine, the cytokine analog, and/or the Fc portion. Where targeting is desired, the chimeric molecule may further comprise an affinity portion (e.g., scFv) that may be covalently bound to the cytokine receptor, the cytokine, the cytokine analog, and/or the Fc portion.

Moreover, it should be appreciated that the Fc portions of two chimeric molecules may associate, for example, via a disulfide bond of cysteine residues in the Fc portions to so form an antibody-like chimeric molecule complex. As will be readily appreciated the first and the second chimeric molecules in such complexes may be identical, or distinct.

In another aspect of the inventive subject matter, the inventors also contemplate a pharmaceutical composition that includes the chimeric molecule or chimeric molecule complex as described herein, typically in combination with a pharmaceutically acceptable carrier (e.g., formulated for injection). Such compositions may then further include one or more cytokines, checkpoint inhibitors, and/or a chemotherapeutic agent.

Therefore, and viewed from a different perspective, the inventors also contemplate a method of treating a person diagnosed with a cancer, in which the pharmaceutical composition presented herein are administered to the person. In addition, the person may further receive chemotherapy, and especially low dose chemotherapy that may be provided on a metronomic schedule. Additionally and/or alternatively, the person may further receive ALT803, a cytokine, and/or a checkpoint inhibitor as part of the treatment. Consequently, the inventors also contemplate use of the chimeric molecule or the chimeric molecule complex in the treatment of a cancer.

In still another aspect of the inventive subject matter, the inventors contemplate a recombinant immunoglobulin protein complex. The recombinant immunoglobulin protein complex includes an Fc domain having first and second Fc portions that are coupled with respective first and second cytokine binding domains having respective first and second target recognition domains. The first and second cytokine binding domains are coupled with first and second cytokines. Most preferably, at least one of the first and second target recognition domains is configured to bind PD-L1. In some embodiments, at least one of the first and second cytokine binding domain is IL-15Rα, or a modified IL-15Rα that increases or decreases interaction with IL-15Rβ or IL-15Rγ.

In some embodiments, at least one of the cytokine is IL-15. In other embodiments, at least one of the first and second target recognition domains is selected from a group consisting of: a PD-L1 antibody, Fab fragment of a PD-L1 antibody, scFv fragment binding to PD-L1. Additionally, in some embodiments, the protein complex is coupled with a carrier protein via the Fc domain. In such embodiments, the carrier protein can be selected from the following: protein A, protein G, protein Z, albumin, refolded albumin.

Still another aspect of the inventive subject matter includes a method of modulating gene expression in an immune competent cell. In this method, a recombinant immunoglobulin protein complex that includes an Fc domain having first and second Fc portions that are coupled with respective first and second cytokine binding domains having respective first and second target recognition domains is provided. The first and second cytokine binding domains are coupled with first and second cytokines. Most preferably, at least one of the first and second target recognition domains is configured to bind PD-L1. Then, the recombinant immunoglobulin protein complex is contacted to the immune competent cell, preferably, at least one of CD4+ T cell, CD8+ T cell, and NK cell, in a dose and schedule effective to modulate gene expression, preferably to increase or decrease mRNA expression of at least two genes at least two fold, and more preferably to increase or decrease mRNA expression of at least three genes at least three fold.

Preferably, the first and second Fc portions form a dimer. In some embodiments, at least one of the first and second cytokine binding domain is IL-15Rα and/or a modified IL-15Rα that increases or decreases interaction with IL-15Rβ or IL-15Rγ. In some embodiments, at least one of the cytokine is IL-15. In other embodiments, at least one of the first and second target recognition domains is selected from a group consisting of: a PD-L1 antibody, Fab fragment of a PD-L1 antibody, scFv fragment binding to PD-L1. Additionally, in some embodiments, the protein complex is coupled with a carrier protein via the Fc domain. In such embodiments, the carrier protein can be selected from the following: protein A, protein G, protein Z, albumin, refolded albumin.

In still another aspect of the inventive subject matter, the inventors contemplate a method of treating a tumor in a patient having the tumor. In this method, a recombinant immunoglobulin protein complex that includes an Fc domain having first and second Fc portions that are coupled with respective first and second cytokine binding domains having respective first and second target recognition domains is provided. The first and second cytokine binding domains are coupled with first and second cytokines. Most preferably, at least one of the first and second target recognition domains is configured to bind PD-L1. Then, the method continues with pre-exposing the recombinant immunoglobulin protein complex to at least one of an immune competent cell and a tumor cell of the tumor, where the pre-exposure of the recombinant immunoglobulin protein complex cell increases effectiveness of antibody-dependent cell cytotoxicity against the tumor.

Preferably, the first and second Fc portions form a dimer. In some embodiments, at least one of the first and second cytokine binding domain is IL-15Rα and/or a modified IL-15Rα that increases or decreases interaction with IL-15Rβ or IL-15Rγ. In some embodiments, at least one of the cytokine is IL-15. In other embodiments, at least one of the first and second target recognition domains is selected from a group consisting of: a PD-L1 antibody, Fab fragment of a PD-L1 antibody, scFv fragment binding to PD-L1. Additionally, in some embodiments, the protein complex is coupled with a carrier protein via the Fc domain. In such embodiments, the carrier protein can be selected from the following: protein A, protein G, protein Z, albumin, refolded albumin.

The inventors contemplate various embodiments for pre-exposing the recombinant immunoglobulin protein complex to tumor cells and/or immune competent cells, preferably at least one of a naïve NK cell and a genetically modified NK cell. In some embodiments, immune competent cells only are pre-exposed to the recombinant immunoglobulin protein complex. In other embodiments, tumor cells only are pre-exposed to the recombinant immunoglobulin protein complex. In still other embodiments, both immune competent cells and tumor cells are pre-exposed to the recombinant immunoglobulin protein complex. In those embodiments, the immune competent cells are preferably pre-exposed to the recombinant immunoglobulin protein complex for at least 12 hours before contacting the tumor, and the tumor cells are preferably pre-exposed to the recombinant immunoglobulin protein complex for at least 30 minutes before contacting the immune competent cells. Optionally, in such embodiments, the immune competent cell is pre-exposed to a CD-16 antagonist before contacting the pre-exposed tumor cell. In some embodiments, the tumor cell is pre-exposed to an EGFR antagonist.

Optionally, the method may further comprise a step of administering the patient at least one of a TGF-β antagonist, an IL-8 antagonist, and a chemokine, which can be CXCL14. It is preferred that the pre-exposed immune competent cell increases secretion of at least one of IFN-γ, TGF-α, IL-6 and IL-8.

Viewed from a different perspective, the inventors also contemplate a method of increasing effectiveness of immunotherapy in a patient having a tumor. In this method, a recombinant immunoglobulin protein complex that includes an Fc domain having first and second Fc portions that are coupled with respective first and second cytokine binding domains having respective first and second target recognition domains is provided. The first and second cytokine binding domains are coupled with first and second cytokines. Most preferably, at least one of the first and second target recognition domains is configured to bind PD-L1. Then, the method continues with pre-exposing the recombinant immunoglobulin protein complex to at least one of an immune competent cell and a tumor cell of the tumor, where the pre-exposure of the recombinant immunoglobulin protein complex cell increases effectiveness of antibody-dependent cell cytotoxicity against the tumor.

Preferably, the first and second Fc portions form a dimer. In some embodiments, at least one of the first and second cytokine binding domain is IL-15Rα and/or a modified IL-15Rα that increases or decreases interaction with IL-15Rβ or IL-15Rγ. In some embodiments, at least one of the cytokine is IL-15. In other embodiments, at least one of the first and second target recognition domains is selected from a group consisting of: a PD-L1 antibody, Fab fragment of a PD-L1 antibody, scFv fragment binding to PD-L1. Additionally, in some embodiments, the protein complex is coupled with a carrier protein via the Fc domain. In such embodiments, the carrier protein can be selected from the following: protein A, protein G, protein Z, albumin, refolded albumin.

The inventors contemplate various embodiments for pre-exposing the recombinant immunoglobulin protein complex to tumor cells and/or immune competent cells, preferably at least one of a naïve NK cell and a genetically modified NK cell. In some embodiments, immune competent cells only are pre-exposed to the recombinant immunoglobulin protein complex. In other embodiments, tumor cells only are pre-exposed to the recombinant immunoglobulin protein complex. In still other embodiments, both immune competent cells and tumor cells are pre-exposed to the recombinant immunoglobulin protein complex. In those embodiments, the immune competent cells are preferably pre-exposed to the recombinant immunoglobulin protein complex for at least 12 hours before contacting the tumor, and the tumor cells are preferably pre-exposed to the recombinant immunoglobulin protein complex for at least 30 minutes before contacting the immune competent cells. Optionally, in such embodiments, the immune competent cell is pre-exposed to a CD-16 antagonist before contacting the pre-exposed tumor cell. In some embodiments, the tumor cell is pre-exposed to an EGFR antagonist.

Furthermore, the inventors also contemplate uses of the recombinant immunoglobulin protein complex described above for modulating gene expression of an immune competent cell, for treating a tumor in a patient having the tumor, and for increasing the effectiveness of immunotherapy in a patient having a tumor.

Various objects, features, aspects and advantages of the inventive subject matter will become more apparent from the following detailed description of preferred embodiments, along with the accompanying drawing.

### BRIEF DESCRIPTION OF THE DRAWING

FIGS. 1A-K illustrates exemplary chimeric molecules and chimeric molecule complexes ((A)-(K)) according to the inventive subject matter.

FIG. 2A illustrates a schematic drawing of another exemplary chimeric molecule having PD-L1 binding domain coupled or fused with IL-15 superagonist.

FIG. 2B-C show graphs of CD4+ or CD8+ T cell proliferation, respectively, upon treatment with the chimeric molecule of FIG. 2A.

FIGS. 3A-B show heat maps of CD4 and CD8 T cell gene expression, respectively, upon treatment with the chimeric molecule of FIG. 2A.

FIG. 4 shows histograms of four phenotypic NK markers showing the change in expression upon treatment of with chimeric molecule of FIG. 2A.

FIG. 5 shows a heat map of gene expression in NK cells upon treatment with the chimeric molecule of FIG. 2A.

FIG. 6A shows a schematic of one pre-exposure with the chimeric molecule of FIG. 2A.

FIGS. 6B-D show graphs of tumor cell lysis in three different tumor cell lines, H441 (lung carcinoma), CaSki (cervical carcinoma), and MDA-MB-231 (breast carcinoma) after pre-exposure with the chimeric molecule as described in FIG. 2A.

FIG. 6E shows a schematic of pre-exposure with the chimeric molecule of FIG. 2A.

FIGS. 6F-H show graphs of tumor cell lysis in three different tumor cell lines, H441 (lung carcinoma), CaSki (cervical carcinoma), and MDA-MB-231 (breast carcinoma) after pre-exposure with the chimeric molecule as described in FIG. 6E.

FIG. 6I shows a schematic of still another pre-exposure with the chimeric molecule of FIG. 2A.

FIGS. 6J-L show graphs of tumor cell lysis in three different tumor cell lines, H441 (lung carcinoma), CaSki (cervical carcinoma), and MDA-MB-231 (breast carcinoma) after pre-exposure with the chimeric molecule as described in FIG. 6I.

FIG. 7A shows a schematic of still another pre-exposure with the chimeric molecule of FIG. 2A.

FIGS. 7B-D show graphs of tumor cell lysis in three different tumor cell lines, H441 (lung carcinoma), CaSki (cervical carcinoma), and MDA-MB-231 (breast carcinoma) after pre-exposure with the chimeric molecule as described in FIG. 7A.

FIG. 7E shows a schematic of still another pre-exposure with the chimeric molecule of FIG. 2A.

FIGS. 7F-H show graphs of tumor cell lysis in three different tumor cell lines, H441 (lung carcinoma), CaSki (cervical carcinoma), and MDA-MB-231 (breast carcinoma) after pre-exposure of the chimeric molecule as described in FIG. 7E.

FIG. 8A shows a schedule of administration of the chimeric molecule of FIG. 2A to a patient having metastatic cancer.

FIG. 8B shows a graph of reduced lung metastases after administering the chimeric molecule of FIG. 2A to the patient in a schedule shown in FIG. 8A.

### DETAILED DESCRIPTION

The inventors now discovered that immune therapy, and particularly T cell- or NK cell-based immune therapy against tumor cells can be significantly improved by modulating T cell or NK cell gene expression and/or by sensitizing tumor cells to antibody-mediated cell-mediated cytotoxicity (ADCC). Such modulation of gene expression and/or sensitization can be achieved by inactivating or binding PD-L1 and/or other upregulated/expressed proteins on the tumor cells/immune competent cells while at the same time providing IL15 stimulatory effect.

Viewed from a different perspective, the inventors discovered that various recombinant protein and/or protein complex having one or more target recognition domains binding PD-L1 and/or other upregulated/expressed proteins on the tumor cells/immune competent cells can be generated such that the recombinant protein and/or protein complex specifically traps, inhibits, or inactivates PD-L1 or and/or other upregulated/expressed proteins. Inhibition of PD-L1 and/or other upregulated/expressed proteins on the tumor cells/immune competent cells in combination with an IL15 stimulatory effect is thought to trigger activation of immune competent cells and/or sensitization of the tumor cells to the ADCC.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻³M, 10⁻⁴M, 10⁻⁵M, 10⁻⁶M, or equal or less than 10⁻⁷M. As used herein, the term “provide” or “providing” refers to and includes any acts of manufacturing, generating, placing, enabling to use, or making ready to use.

### Chimeric Molecules

Thus, the inventors contemplate that one inventive subject matter is directed to various chimeric molecules and complexes formed by such chimeric molecules. Most typically, the chimeric molecules will include at least an Fc portion of an antibody and an immunomodulatory portion, in which the immunomodulatory portion comprises at least two immune effectors. Due to the presence of the Fc portion, chimeric molecules contemplated herein will be able to associate with each other via a disulfide bridge as is common in antibodies, and so formed chimeric molecule complexes may comprise identical or non-identical chimeric molecules.

It should be especially appreciated that the multimeric immune effectors contemplated herein will have substantially increased biologically activity as compared to entities in which such effectors are present in a single copy. Moreover, and as is further described in more detail below, higher copy numbers of immune effectors may also provide additional effects as is particularly the case with PD-L1 and PD-1. Still further, contemplated chimeric molecules and complexes thereof may provide additional therapeutic effects due to the presence of the Fc portion that advantageously stimulates ADCC. In addition, where IL15 or IL15 receptor portions are present in the chimeric molecules, these portions will additionally attract and activate T cells and NK cells. Therefore, it should be appreciated that the chimeric molecules and complexes thereof will have multiple therapeutic effects, and may in some circumstances even be viewed as stimulators of endogenous ADCC (antibody-dependent cell-mediated cytotoxicity).

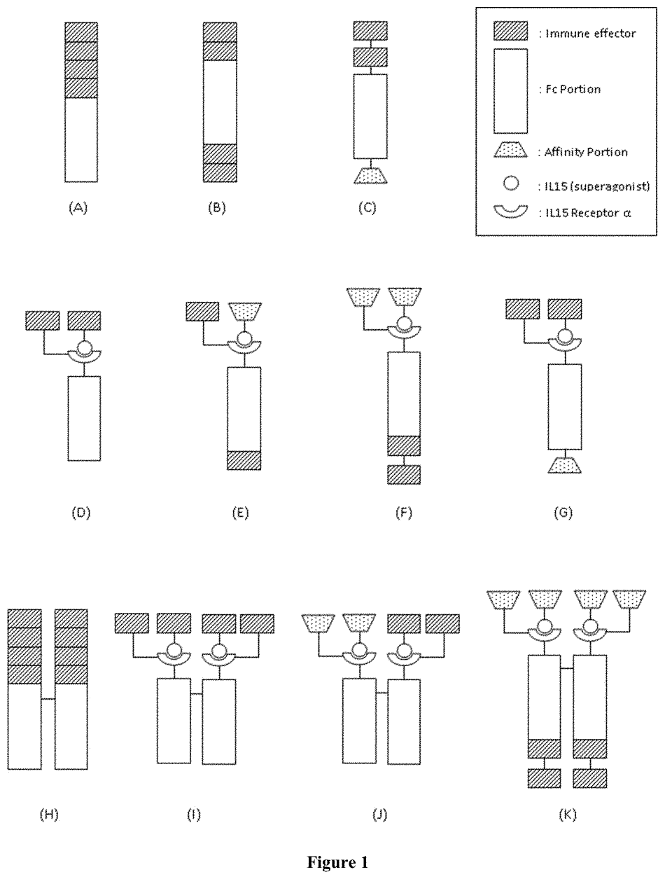

FIGS. 1A-K illustrate various exemplary configurations of contemplated chimeric molecules and complexes. For example, in FIG. 1A, the N-terminus of an Fc portion of an antibody is covalently coupled to the C-terminus of an immunomodulatory portion that is configured as a concatemeric sequence of four immune effectors. No spacer portions are used in this construct. For example, such construct could be useful in the preparation of a chimeric molecule (or molecule complex) in which the four immune effectors are co-stimulator ligands such as 4-1BB, OX40L, and/or GITRL, and in which the Fc portion is from a human IgG. It should be noted that not all of the immune effectors need to be in a single chain, but that the immune effectors may be linked both to the N- and C-terminus of the Fc portion as can be seen from FIG. 1B. Where desired or sterically required, the immune effectors may also be separated by a typically flexible linker, for example, a (G₄S)₃ linker, a G₈ linker, a G₆ linker, a (EAAAK)ₙ linker with n=1-3, a PAPAP linker, or a GFLG linker as is schematically illustrated in FIG. 1C. Here, the immune effectors are coupled to each other by a first linker, and coupled to the Fc portion by a second linker. In this construct, an additional affinity portion (e.g., scFv) is also coupled to the other terminus of the Fc portion to enable targeting the chimeric molecule to a specific target.

Where the chimeric molecule is designed to include an IL15 or IL15 superagonist (e.g., a N72D mutant of IL15) in association with an α-chain of the IL15 receptor, contemplated chimeric molecules may be designed as shown in exemplary structures FIGS. 1D-G. Here, the Fc portion of the antibody is covalently bound to the α-chain of the IL15 receptor, and at least one immune effector is covalently bound to the α-chain of the IL15 receptor. Using the specific binding of IL15 to the α-chain of the IL15 receptor, another chimeric molecule comprising IL15 and at least one immune effector can be (typically non-covalently) coupled to the chimeric molecule as exemplarily shown in FIG. 1D. Notably, such molecule will provide the benefits of the Fc portion and the IL15/IL15 receptor, as well as a desired immune modulatory effect via the immunomodulatory portion. It should also be noted that the immune effectors need not be concatemeric, but can instead reside at distinct portions of the chimeric molecule as is shown in FIG. 1E. Alternatively, one or more affinity portions may be implemented together with multiple immune effectors as is depicted in FIG. 1F, where multiple, possibly distinct affinity moieties are used for targeting, and in FIG. 1G where multiple immune effectors are used with one affinity portion.

As will be readily appreciated by the PHOSITA, two identical or distinct Fc portions may associate to form a chimeric molecule complex as is schematically depicted in the homodimers as shown in FIGS. 1H-K, and the heterodimer shown in FIG. 1J. Of course, it should be appreciated that there are more possible combinations of chimeric molecules shown in FIGS. 1A-G than those shown in FIGS. 1H-K, and all possible combinations are expressly contemplated herein.

More specifically, in one exemplary aspect of the inventive subject matter, a chimeric molecule complex may be formed from two identical chimeric molecules, wherein each chimeric molecule includes an Fc portion of a human IgG. Covalently coupled to the Fc portion is the alpha receptor chain of the IL-15 receptor (or an IL-15 binding fragment thereof), to which an scFv portion or an immune effector is covalently bound. Most preferably, where an scFv is employed, the scFv portion has binding affinity to PD-L1. On the other hand where immune effectors are employed, the immune effector is PD-L1, preferably present in at least two, three, or four copies. In this example, the alpha receptor chain of the IL15 receptor further binds IL15 (or an IL15 superagonist) to which an scFv portion or an immune effector is covalently bound. As above, where an scFv is employed, the scFv portion has binding affinity to PD-L1. On the other hand where immune effectors are used, the immune effector is PD-L1, preferably present in at least two, three, or four copies.

Such exemplary construct is thought to have several notable advantages. Where the construct has an scFv that binds PD-L1, the construct is specifically targeted towards cancer cells that express PD-L1 as a means to protect themselves from ADCC or cytotoxic attack by immune competent cells. In addition, the presence of the IL-15 or IL-15 superagonist will advantageously attract and activate T cells and NK cells, while the presence of the Fc portion facilitates ADCC. Moreover, it should be appreciated such construct is especially beneficial in a therapy of a tumor that does not (yet) express PD-L1 as defense mechanism. In such therapy, a first step will include an induction of an immune reaction against the tumor (e.g., radiation, chemotherapy, and/or cell or vaccine type neoepitope-based treatments). In a second step, where the tumor now attempts evasion of an immune response by down-regulating immune competent cells (e.g., T cells), the chimeric molecule is now administered and specifically targets those cells that attempt immune evasion.

Similarly, where the tumor cells have or are about to evade an immune response by activating the PD-1/PD-L1 pathway, chimeric molecules may be administered that have multiple copies of PD-L1 or PD-1 as immune effectors. Once more, such therapeutics will not only provide a desired effect by way of interference with the PD-1/PD-L1 pathway, but also attract and activate T cells and NK cells, while the presence of the Fc portion facilitates ADCC. Viewed from a different perspective, it should be appreciated that biological effects of the components in the chimeric molecules and complexes will be amplified by presence of multiple immune effectors. In at least some cases, effects may also be also reversed as, for example, with PD-L1. PD-L1 is typically presented by a cancer cell as an inhibitory co-stimulatory molecule. However, higher quantities, and especially of the extracellular portion of both PD-1 and PD-L1 have been shown to interfere with PD-1/PD-L1 signaling. Thus, a system that targets PD-1/PD-L1 signaling may include multimeric PD-L1 and/or multimeric PD-1 portions as immune effectors or include an affinity portion (e.g., scFv, etc.) targeting and binding PD-L1. In that case, the affinity portion will also act as an immune effector as PD-1/PD-L1 signaling is interrupted.

Of course, it should be appreciated that contemplated compounds, compositions, and methods need not be limited those in which the Fc portion is obtained from human IgG. Indeed, all known Fc portions and other antibody fragments (e.g., Fab, Fab′, F(ab)₂, etc.) are deemed suitable for use herein, and therefore include Fc portions form IgG, IgM, IgE, and IgA. Moreover, where an ADCC or other Fc dependent reaction is not desired, the Fc portion may be genetically modified to abrogate ADCC. However, it is generally preferred that the Fc portion will include sufficient sequence to allow (i) formation of dimers via a disulfide bond, (ii) binding to protein A or protein G, and/or (iii) binding to the Sudlow-II domain of albumin, and especially refolded albumin (which may be loaded with a drug such as a taxane). Therefore, the inventors also expressly contemplate all chimeric molecule complexes as described herein in which two Fc portions have associated (typically via a disulfide bond), and all compositions in which the Fc portion has bound to another protein (e.g., albumin, protein A, protein G, etc.).

Likewise, with respect to the particular immune effector it is contemplated that all protein molecules are deemed suitable that modulate (up- and/or down-regulate) an immune response, typically by way of specific interaction with one or more ligand or receptor of a co-stimulatory pathway (activating or suppressing). For example, especially preferred immune effectors include 4-1BBL, OX40L, and/or GITRL. Moreover, especially preferred immune effectors include PD-1, PD-L1, binders of PD-1, binders of PD-L1, as well as PD-L2, and binders of PD-L2. With respect to these immune effectors, it is particularly preferred that such effectors will be limited to the extracellular domains (or soluble forms and/or splice variants) but retain regulatory activity. For example, the extracellular domain of human PD-L1 includes amino acids 1-238 with intact binding activity to PD-1. Similarly, in murine PD-L1, amino acids 1-237 denote the extracellular portion with retained binding activity to PD-1. With further respect to the number and arrangement of the immune effectors it is contemplated that the chimeric molecule comprises at least two, or at least three, or at least four immune effectors, which may be in concatemeric arrangement, or otherwise distributed on the chimeric molecule. Viewed from a different perspective it is contemplated that the number of immune effectors is at least such that the immune modifying activity of a plurality of effectors is greater than that of a single effector, regardless of their particular arrangement. For example, four OX40 ligands may be serially (concatemeric) arranged at the N-terminus of an Fc, while in other aspects, two 4-1BB ligands may be present at two different portions of a chimeric molecule complex (e.g., one covalently bound to IL-15 receptor alpha chain, and another covalently bound to IL15, where IL15 is bound to the IL-15 receptor alpha chain, etc.). Alternatively, it is contemplated that two immune effectors on the chimeric molecule can be two distinct immune effectors, which can be arranged linearly or distributed separately. For example, N-terminus of an Fc can be coupled with one OX40 ligand and one 4-1BB ligand that are linearly or sequentially placed (e.g., encoded by a single nucleic acid sequence) via a linker. In another example, one of N-terminus of Fcs can be coupled with one or more OX40 ligand and another of N-terminus of Fcs can be coupled with one or more 4-1BB ligand.

Suitable examples of PD-L1 include human PD-L1 and the corresponding ligands of various mammals, including chimpanzee, cynomolgus monkey, mouse, rat, guinea pig, dog, and pig. Particularly, human PD-L1 may have an amino acid sequence that is identified by GenBank Accession. No. NP_0054862 or NP_0054862.1, while the murine PD-L1 may have an amino acid sequence that is identified by GenBank Accession. No. NP_068693 or NP_068693.1. Likewise, human PD-L1 cDNA is identified by GenBank Accession. No. NM_014143, NM_014143.1 or NM_014143.2, while murine PD-L1 cDNA is identified by GenBank Accession. No. NM_021893, NM_021893.1 or NM_021893.2.

Where the chimeric molecule includes the α-chain of the IL-15 receptor, all forms of such receptor are deemed suitable for use herein. However, especially preferred forms will retain the ability to bind IL-15 or an IL-15 superagonist and only present the α-chain. There are various fusion constructs between the IL-15 receptor α-chain and an Fc portion known (e.g., Oncotarget, Vol. 7, No. 13, 2016, p. 16130-16146) and all manners of preparing such constructs are deemed suitable for use herein. Likewise, there are numerous IL-15 and IL-15 superagonists known in the art (see e.g., J Immunol 2009; 183:3598-3607), and once more, all of those are deemed suitable for use herein.

Still further, where the chimeric molecule includes an affinity portion, it is preferred that the affinity portion is a scFv and binds a tumor associated antigen, a tumor specific antigen, or a patient and tumor specific neoepitope (preferably MHC matched to the patient). In further aspects of the inventive subject matter, the scFv may also bind to a peptide or protein that is over-expressed in the tumor of the patient. Alternatively, suitable affinity portions may also include proteins that were obtained by affinity maturation (e.g., using phage display) or by RNA display. Most typically, the affinity portion will form part of the polypeptide of the chimeric molecule.

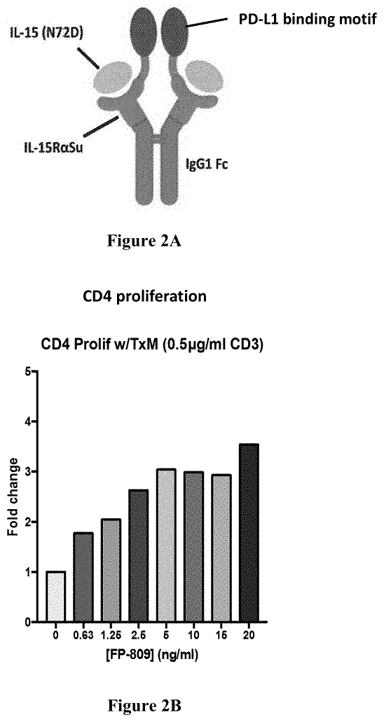

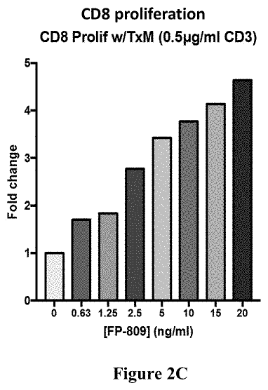

One especially preferred embodiment of chimeric molecule includes a recombinant immunoglobulin protein complex that has one or more PD-L1 binding domain as shown in FIG. 2A. The recombinant immunoglobulin protein complex includes an Fc domain that has two Fc portions, each of which is coupled with a cytokine binding domains. Preferably, each of the two Fc portions includes a hydrophobic interface to interact with each other to form a dimer. The hydrophobic interface includes at least 10 amino acids, at least 15 amino acids, at least 20 amino acids, at least 25 amino acids, similar to Fc domain of human immunoglobulin G. Alternatively, at least one of the two Fc portions can be engineered such that the single Fc portion can be stable and soluble without forming a dimer with another Fc portion.

Each of the Fc portions is typically coupled with a cytokine binding domain at the N-terminus of the Fc portion, and each of the cytokine binding domains is bound to a ligand (a cytokine molecule). Any suitable cytokine binding domains are contemplated, including, but not limited to interleukin-15 (IL-15) binding protein (e.g., a full length or an IL-15 binding motif of IL-15 receptor α, etc.), CD25 (IL-2 binding protein), IL-4 receptor α, IL-13 receptor α, or IL-21 receptor α. Of course, the preferred cytokine is the corresponding high affinity ligand for the cytokine binding domains, for example, IL-15 for a full length or an IL-15 binding motif of IL-15 receptor α, and IL-2 for CD25. In some embodiments, the cytokine binding domain is directly coupled to the N-terminus of Fc portion. In other embodiments, the cytokine binding domain is coupled to the N-terminus of Fc portion via a linker or a spacer, which is typically between 3-30 amino acids, preferably between 5-20 amino acids, more preferably between 5-15 amino acids. The inventors contemplate that glycine-rich sequences (e.g., gly-gly-ser-gly-gly, etc.) are preferred to provide structural flexibility between the Fc portion and the cytokine binding domain, especially when the cytokine binding domain is bulky and may provide steric hindrance to other nearby domains. The inventors also contemplate that in some embodiments, at least one of the cytokine binding domains and cytokines coupled to those can be substituted with other pairs of molecules coupled by high affinity protein-protein interaction. For example, the pairs of molecules may include an enzyme (preferably inactive enzyme)-peptide substrate, or a toxin receptor-inactive toxin (e.g., recombinant fusion toxin).

In some embodiments, the cytokine binding domain can be modified to increase the biological effect of cytokine binding to the binding domain. For example, where the cytokine binding domain is IL-15 receptor α (IL-15Rα), and the cytokine is IL-15, the IL-15Rα:IL-15 complex can trans-interact with a membrane-bound IL-15 receptor β and IL-15 receptor γ to elicit IL-15-mediated signaling cascade in the cell expressing IL-15 receptor β and IL-15 receptor γ. Thus, the inventors contemplate that a portion of the IL-15Rα can be modified so that the interaction between the IL-15Rα: IL-15 and IL-15 receptor β or IL-15 receptor γ can be enhanced. A particularly preferred cytokine is a superagonist version, especially in combination with ALT-80.

The inventors contemplate that one or more cytokine binding domains, more preferably each of the cytokine binding domains are coupled with one or more binding motif that is configured to bind PD-L1. In some embodiments, the cytokine binding domain is directly coupled to PD-L1 binding motif. In other embodiments, the cytokine binding domain is coupled to PD-L1 binding motif via a linker or a spacer, which is typically between 3-30 amino acids, preferably between 5-20 amino acids, more preferably between 5-15 amino acids. Similar to coupling between the Fc portion and the cytokine binding domain, glycine-rich sequences (e.g., gly-gly-ser-gly-gly, etc.) as a linker or a spacer are preferred to provide structural flexibility between the cytokine binding domain (or cytokine) and the target recognition domain, especially when one or more target recognition domain is bulky and may provide steric hindrance to other target recognition domains.

Any suitable PD-L1 binding motifs that can be linked to the cytokine binding domain of the recombinant immunoglobulin protein complex without providing significant steric hindrance or functional defect are contemplated. Thus, suitable PD-L1 binding motif includes a whole PD-L1 antibody, a portion of PD-L1 antibody (e.g., one of Fab domain or two Fab domains, etc.), an scFv fragment binding to PD-L1. In some embodiments, two cytokine binding domain of the recombinant immunoglobulin protein complex can be associated with two different types of PD-L1 binding motif (e.g., one with Fab domain of PD-L1 antibody and another with scFv fragment binding to PD-L1, etc.). In other embodiments, it is also contemplated that two cytokine binding domain of the recombinant immunoglobulin protein complex can be associated with same type of PD-L1 binding motif.

Contemplated molecules and complexes can be prepared using various known methods. For example, it is contemplated that chimeric molecule complexes can be expressed in several mammalian, yeast, or even bacterial cells from an appropriately constructed nucleic acid (DNA or RNA). Preferably, the components of the recombinant immunoglobulin protein complex may be encoded by one or more recombinant nucleic acids. For example, the recombinant nucleic acid may include at least two nucleic acid segments (a sequence element): a first nucleic acid segment encoding in a single reading frame an Fc portion, a cytokine binding domain, and a PD-L1 binding motif; and a second nucleic acid segment encoding in a single reading frame a cytokine and another target recognition domain (preferably a type of PD-L1 binding motif). In other examples, the two nucleic acid segments are in the same reading frame such that two nucleic acid segments can be translated into a single protein having two peptide segments under the same promoter. In case, the inventors contemplate that the first and second nucleic acid segments are spaced with a spacer sequence (e.g., a nucleic acid sequence encoding a linker or a spacer of at least 10 amino acids, 15 amino acids, 20 amino acids, etc.). In other embodiments, the two nucleic acid segments may be transcribed separately into two distinct peptides. In still other embodiments, the two nucleic acid segments are present in the same reading frame, but separated by nucleic acid sequences encoding a type of 2A self-cleaving peptide (2A). As used herein, 2A self-cleaving peptide (2A) refers any peptide sequences that can provide a translational effect known as “stop-go” or “stop-carry” such that two sub-segments in the same mRNA fragments can be translated into two separate and distinct peptides. Any suitable types of 2A peptide sequences are contemplated, including porcine teschovirus-1 2A (P2A), thosea asigna virus 2A (T2A), equine rhinitis A virus 2A (E2A), foot and mouth disease virus 2A (F2A), cytoplasmic polyhedrosis virus (BmCPV 2A), and flacherie virus (BmIFV 2A)

Most preferably, the chimeric molecules will be expressed as a single polypeptide chain. However, individual components of the chimeric molecule complexes may also be expressed individually, and then fused together after expression. Among other advantages, purification of contemplated chimeric molecules is greatly simplified due to the presence of the Fc portion and its ability to specifically and avidly bind to certain proteins (e.g., Protein A or G) or binding to the Sudlow II domain in albumin. When forming complexes, it is contemplated that first and second chimeric molecules can be made in separate batches.

Alternatively or additionally, the chimeric molecule can be further coupled with an anchor molecule such that the chimeric molecule can be coupled to a carrier molecule via an anchor molecule. For example, where the carrier protein is an albumin, the anchor molecule can be a hydrophobic peptide or glycolipids in any suitable size (e.g., in a length of at least 10 amino acids, 15 amino acids, 20 amino acids, 30 amino acids, etc.) to fit in one of Sudlow's site I and II of the albumin or any other hydrophobic area of the albumin. Other contemplated carrier molecules include, but not limited to, a nanoparticle (e.g., quantum dots, gold nanoparticles, magnetic nanoparticles, nanotubes, polymeric nanoparticles, dendrimers, etc.), or a bead (e.g., polystyrene bead, latex bead, dynabead, etc.). Preferably, the nanoparticle and/or beads have a dimension below 1 μm, preferably below 100 nm.

Pre-Exposure of Chimeric Molecule to Immune Competent Cells and/or Tumor Cells

The inventors found that immune competent cells treated with or exposed to one of chimeric molecule (FP-809, a chimeric molecule having Fc domain of IL-15 superagonist (ALT-803) fused with two scFv fragments binding to PD-L1, as shown in FIG. 2A), can trigger proliferation, activation, and/or differential gene expression in the immune competent cell. As used herein, immune competent cells refer any immune cells that can elicit immune response upon recognition or in the presence of tumor cells. Thus, the immune competent cells preferably include T cells (e.g., CD4+ T cells, CD8+ T cells, Treg cells, etc.), NK cells, NKT cells, antigen presenting cells (e.g., dendritic cells, etc.).

For example, as shown in FIGS. 2B-C, CD4+ and CD8+ T cells significantly proliferate upon exposure to FP-809. In this experiment, CD4 and CD8 T cells from two healthy donors were used in proliferation assays with plate-bound anti-CD3. T cells were added to the assay in the presence of increasing concentrations of FP-809, where the concentration of FP-809 is in a physiological or physiologically acceptable range. Fold change represents the change from untreated (0 ng/ml), anti-CD3 stimulated T cells. Addition of FP-809 substantially increased the proliferation of CD4 T cells (about 3-fold, FIG. 2B) and CD8 T cells (about and more than 4-fold, FIG. 2C) in a dose dependent manner.

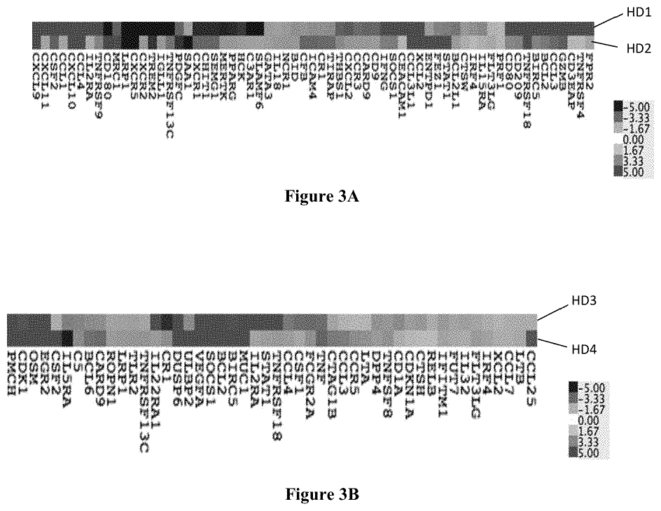

Further, CD4+ and CD8+ T cells showed significant changes in gene expression upon exposure to FP-809. CD4 and CD8 T cells from two healthy donors were incubated with or without FP-809 for 24h prior to RNA isolation for NanoString analysis using the nCounter PanCancer Immune Profiling Panel of 770 immune related genes. As shown in heat maps in FIGS. 3A-B, CD4+ cells from two different donors (FIG. 3A) show consistent upregulations (at least two fold) in a set of genes (shown in red), and downregulations (at least two fold) in another set of genes (shown in blue). As shown in Table 1, listing exemplary genes differentially expressed in CD4 and CD8 T cells, upon treatment with the chimeric molecule of FIG. 2A, out of 770 genes, about 8.2% (63 genes), 3.6% (28 genes), 0.1% (1 gene) showed at least 2 fold, at least 3 fold, at least 10 fold changes, respectively in CD4+ cells, and about 6.4% (49 genes), 2.2% (17 genes), 0.3% (2 genes) showed at least 2 fold, at least 3 fold, at least 10 fold changes, respectively in CD8+ cells compared to untreated/unexposed CD4+ and CD8+ T cells. In addition, CD4+ and CD8+ T cells exposed to FP-809 also showed increase of several cytokines. CD4+ and CD8+ T cells were isolated from two healthy donors. Isolated cells and an MDA-MB-231 neoantigen-specific T cell line were incubated with or without FP-809 at a concentration of 37.5 ng/ml for 24 hrs. Cell concentrations were kept consistent between treated and untreated samples at 1×10⁶ cells/ml. Supernatants were collected and the cytokine concentration secreted from the cells were analyzed by a multiplex assay. As shown in Table 2, in at least one CD4+ T cell samples, the amount of four different cytokines, IFN-γ, TGF-α, IL-6 and IL-8, were all significantly increased, from at least 2.5 fold (IL-8) to over 700 fold (IFN-γ). Yet, such increase of cytokines was not apparent in CD8+ T cells and MDA-MB-231 neoantigen-specific T cell line.

TABLE 1
Genes Differentially Expressed vs. Untreated Control

| | CD4 | | | | | | CD8 | | | | | |
|---|---|---|---|---|---|---|---|---|---|---|---|---|
| | Count (% of 770) | | | | | | Count (% of 770) | | | | | |
| | ≥2 fold | | ≥3 fold | | ≥10 fold | | ≥2 fold | | ≥3 fold | | ≥10 fold | |
| Positive or | 63 | (8.2) | 28 | (3.6) | 1 | (0.1) | 49 | (6.4) | 17 | (2.2) | 2 | (0.3) |
| Negative Change | | | | | | | | | | | | |
| Increased Expression | 33 | (4.3) | 19 | (2.5) | 0 | (0) | 39 | (5.1) | 16 | (2.1) | 2 | (0.3) |
| Decreased Expression | 30 | (3.9) | 9 | (1.2) | 1 | (0.1) | 10 | (1.3) | 1 | (0.1) | 0 | (0) |

TABLE 2

| | | IFNγ | TNFα | IL-6 | IL-8 |
|---|---|---|---|---|---|
| CD4 T cells | | | | | |
| HD1 | Untreated | <1.66 | 5.42 | 27.79 | 783.74 |
| | FP-809 | 1204.83 | 34.03 | 91.03 | 2175.66 |
| HD2 | Untreated | 3.23 | 2.17 | 1.34 | 296.24 |
| | FP-809 | 6.92 | 2.39 | 1.44 | 299.19 |
| CD8 T cells | | | | | |
| HD3 | Untreated | 3.04 | 0.54 | <0.78 | 2.86 |
| | FP-809 | 4.42 | 1.89 | <0.78 | 2.50 |
| HD4 | Untreated | <1.66 | <0.39 | <0.78 | 3.40 |
| | FP-809 | 4.96 | 0.45 | <0.78 | 3.40 |
| Ag-specific T cells | | | | | |
| | Untreated | 543.25 | 58.88 | 203.49 | 3002.69 |
| | FP-809 | 741.37 | 69.30 | 210.07 | 3257.15 |

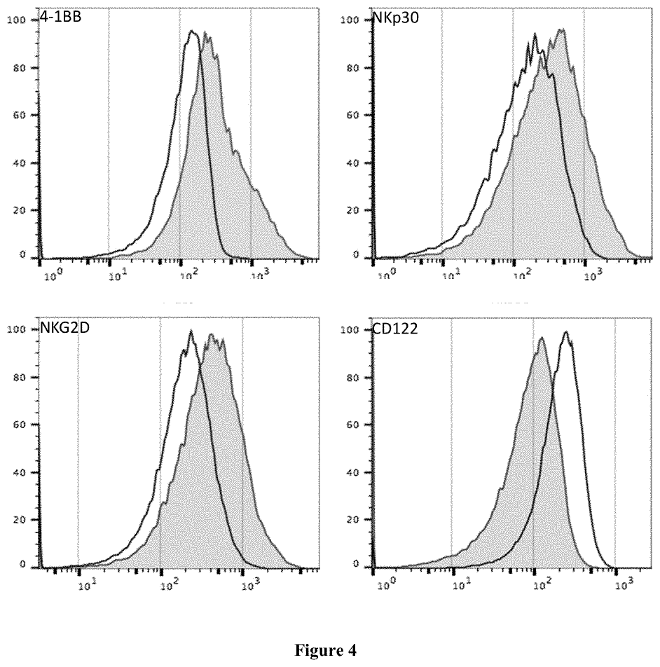

The inventors also found that gene expression in NK cells could also be modulated by exposure to FP-809, and some marker protein expression on the surface of NK cells can be also modulated upon exposure to FP-809. FIG. 4 shows several marker proteins that express substantially differently in NK cells treated with FP-809. In this experiment, healthy donor NK cells were incubated with or without FP-809 for 24 hrs before being stained for multicolor flow cytometry. Table 3 shows markers with increased or decreased expression after FP-809 treatment (listing exemplary markers with increased or decreased expression upon treatment with the chimeric molecule of FIG. 2A). For example, while less than 0.2% of untreated NK cells express 4-1BB protein, almost 10% (9.91%) of NK cells express 4-1BB protein after 24 hour exposure to FP-809. For other example, while more than 28% of untreated NK cells express CD122 (IL-2Rβ), only 2% of NK cells treated with FP-809 express CD122, indicating expression of CD122 is substantially downregulated by FP-809 treatment. FIGS. 4B-E show representative histograms of four phenotypic NK markers showing the change in expression between untreated cells (blue outline) and FP-809 treated cells (red shaded). Some marker proteins showed no significant difference in the expression level between untreated NK cells and treated NK cells, and those markers include NKG2A, NKp46, CD158a, CD16, CD40L, FAS-L, and 2B4.

TABLE 3

| | Untreated | FP-809 |
|---|---|---|
| Marker | (% of NK) | (% of NK) |
| CD25 | 0.00 | 5.76 |
| PD-L1 | 0.47 | 2.23 |
| 4-1BB | 0.19 | 9.91 |
| Tim3 | 0.08 | 0.15 |
| CD56 | 50.59 | 73.61 |
| CD95 | 0.35 | 1.45 |
| NKp30 | 67.42 | 82.71 |
| CD11a | 4.25 | 11.97 |
| CD27 | 6.49 | 12.61 |
| NKG2D | 49.20 | 82.40 |
| NKp44 | 2.73 | 15.42 |
| TRAIL | 0.99 | 14.37 |
| Granzyme B | 66.13 | 91.61 |
| Ki-67 | 6.38 | 12.90 |
| Perforin | 57.02 | 76.68 |
| CD122 | 28.21 | 2.09 |

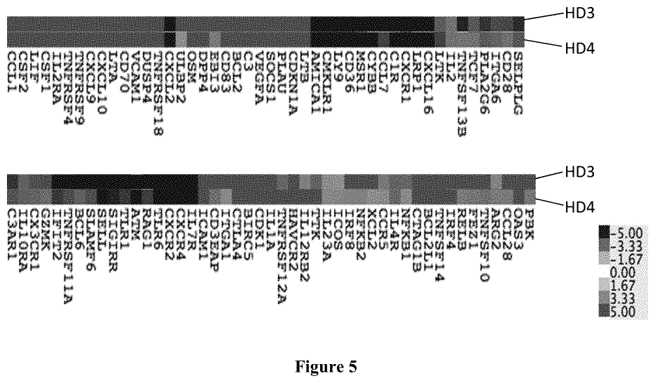

Similar to CD4+ and/or CD8+ T cells, NK cells showed differential gene expression upon exposure to FP-809. Healthy donor NK cells were incubated with or without FP-809 for 24 hrs prior to RNA isolation for NanoString analysis using the nCounter PanCancer Immune Profiling Panel of 770 immune related genes. As shown in heat maps in FIG. 5, NK cells from two different donors show consistent upregulations (at least about two fold or more (1.67 fold-5 fold) in a set of genes (shown in red), and downregulations (at least about two fold or more (1.67 fold-5 fold) in another set of genes (shown in blue). As shown in Table 4 (listing exemplary genes differentially expressed in NK cells, upon treatment with the chimeric molecule of FIG. 2A), out of 770 genes, about 21.4% (165 genes), 12.1% (93 genes), 5.2% (40 gene) showed at least 2 fold, at least 3 fold, at least 10 fold changes, respectively in NK cells.

TABLE 4
Genes Differentially Expressed vs. Untreated Control

| | NK | | | |
|---|---|---|---|---|
| | Count (% of 770) | | | |
| | ≥2 fold | | ≥3 fold | ≥10 fold |
| Positive or Negative Change | 165 | (21.4) | 93 (12.1) | 40 (5.2) |
| Increased Expression | 96 | (12.5) | 56 (7.3) | 28 (3.6) |
| Decreased Expression | 69 | (9.0) | 37 (4.8) | 12 (1.6) |

In addition, NK cells exposed to FP-809 also showed increase of several cytokines. NK cells were isolated from two healthy donors and incubated with or without FP-809 at a concentration of 37.5 ng/ml for 24 hrs. Cell concentrations were kept consistent between treated and untreated samples at 1×10⁶ cells/ml. Supernatants were collected and the cytokine concentration secreted from the cells were analyzed by a multiplex assay. As shown in Table 5, NK cells from two donors treated with FP-809 showed consistent increase of the amount of four different cytokines, IFN-γ, TGF-α, IL-6 and IL-8, from almost at least 2 fold (IL-8) to over 500 fold (IFN-γ).

TABLE 5
NK cell Cytokine Secretion (pg/ml)

| | | IFNγ | TNFα | IL-6 | IL-8 |
|---|---|---|---|---|---|
| HD3 | Untreated | <1.66 | 0.84 | 2.96 | 148.76 |
| | FP-809 | 4688.72 | 43.05 | 18.42 | 342.49 |
| HD4 | Untreated | <1.66 | 0.46 | <0.78 | 8.66 |
| | FP-809 | 528.53 | 14.27 | 0.95 | 25.50 |

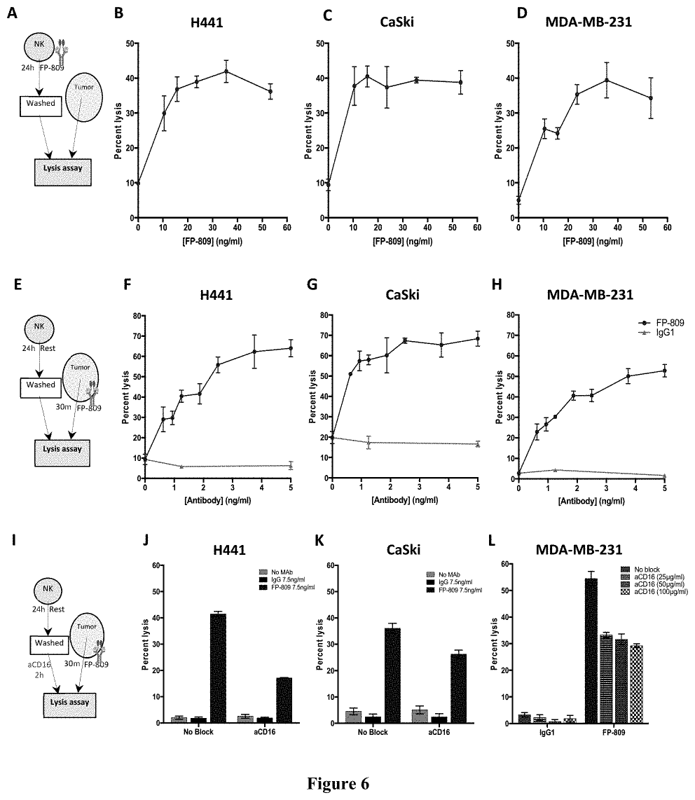

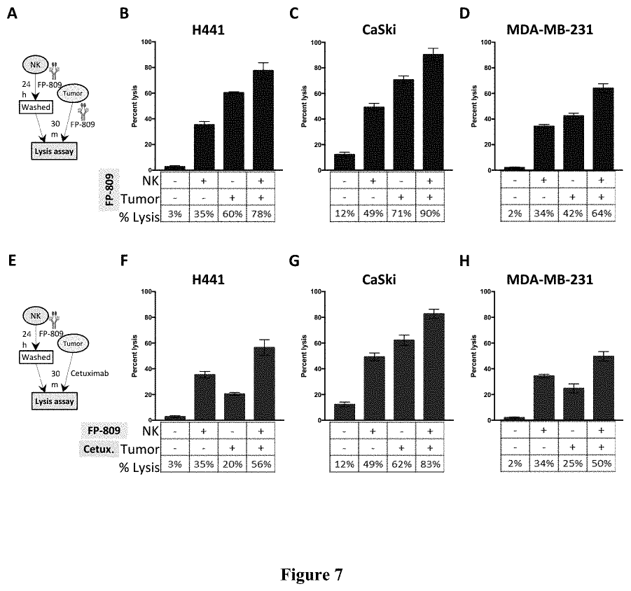

The inventors contemplate that blocking PD-L1 activity as ligand in the tumor cell and/or inducing activation and/or cytokine release by NK cells upon exposure to FP-809 may increase the tumor cell lysis by NK cells when the tumor cell contacts NK cells. In the first experiment, as schematically described in FIG. 6A, NK cells from were pre-exposed or incubated with FP-809 at increasing doses in a range of 0-60 ng/ml for 24 hours, then washed to reduce direct effect of FP-809 to the tumor cell. Then, the pre-exposed (or treated) NK cells were co-incubated (contacted) with tumor cell. All tumor lysis assays (as described in FIGS. 6-7) were performed using as targets: H441 (lung carcinoma, 99.1% PD-L1⁺), CaSki (cervical carcinoma, 78.7% PD-L1⁺), and MDA-MB-231 (breast carcinoma, 66.5% PD-L1⁺) at a 10:1 effector: target (E:T) ratio with multiple donors. Results from one representative donor are shown for each experiment. As shown in FIG. 6B-D, cell lysis percentages of three different types of tumor cells were significantly increased by pre-exposing NK cells to FP-809.

Next, the inventors examined whether exposure of FP-809 only to tumor cells may affect the cell lysis by NK cells. In this experiment, as schematically described in FIG. 6E, NK cells were pre-incubated without FP-809 for 24 hours and washed. Tumor cells (H441, CaSki, MDA-MB-231) were exposed either IgG1 control (grey line) or FP-809 (blue line) at concentrations up to 5 ng/ml for 30 min before co-incubating with washed NK cells. As shown in FIG. 6F-H, cell lysis percentages of three different types of tumor cells were significantly increased by pre-exposing those tumor cells to FP-809.

The inventors then examined whether the effect of FP-809 on the tumor cell to increase cell lysis is via CD-16-mediated signaling pathway in the NK cells, which induces ADCC. In this experiment, as schematically described in FIG. 6I, NK cells were pre-incubated without FP-809 for 24 hours and washed. Then, the NK cells were treated with anti-CD16 (25 μg/ml, 50 μg/ml, or 100 μg/ml) for 2 hours prior to the lysis assay. Tumor cells were exposed to no monoclonal antibody (MAb) control, IgG1 control, or FP-809 at concentrations about 7.5 ng/ml for 30 min before co-incubating with anti-CD16 treated NK cells. As shown in FIGS. 6J-L, while FP-809 increase the cell lysis percentages in all three tumor cell types, treatment of NK cells with anti-CD16 antibody significantly reduces the effect of FP-809. For FIGS. 6J-K, NK cells treated with anti-CD16 antibody at a concentration of 25 μg/ml. For FIG. 6L, NK cells were treated with anti-CD16 antibody at higher concentration (25 μg/ml, 50 μg/ml, or 100 μg/ml) and MDA-MB-231 cells were exposed to FP-809 at a concentration of 10 ng/ml.

Table 6 summarizes the cell lysis percentages in three different tumor cell lines with or without anti-CD16 antibody treatment to the NK cells, which clearly shows that anti-CD16 antibody treatment to the NK cells could reduce the effect of FP-809 on the cell lysis. The percent lysis of three target tumor cell lines by NK cells with or without anti-CD16 blocking (25 μg/ml, 2h). Tumor lysis assays were performed in the presence of no MAb, IgG1 (7.5 ng/ml), or FP-809 (1.8, 3.75, or 7.5 ng/ml). Results shown are the average % lysis (standard deviation) for triplicate wells. Similar results were seen with an additional donor. For example, for H441 cells, with 7.5 ng/ml treatment of FP-809, cell lysis percentage was increased from 2% (no MAb) to 41.5%. Such increase of cell lysis could be substantially inhibited by anti-CD16 blocking such that the cell lysis percentage could increase only to 17.1%, which is about 2.5 fold lower than without anti-CD16 blocking.

TABLE 6

| | | | % Lysis | | % Lysis | |
|---|---|---|---|---|---|---|
| | [Antibody] (ng/ml) | | (−aCD16) | | (+aCD16) | |
| H441 | | | Avg % (SD) | | Avg % (SD) | |
| | No MAb | 0 | 2.0 | (0.6) | 2.6 | (0.7) |
| | IgG1 | 10 | 1.8 | (0.5) | 1.9 | (0.3) |
| | FP-809 | 1.80 | 20.4 | (3.2) | 11.6 | (1.1) |
| | FP-809 | 3.75 | 32.4 | (0.6) | 17.6 | (0.8) |
| | FP-809 | 7.50 | 41.5 | (1.0) | 17.1 | (0.1) |
| CaSki | | | | | | |
| | No MAb | 0 | 4.5 | (1.3) | 5.1 | (1.5) |
| | IgG1 | 10 | 2.5 | (1.0) | 2.4 | (1.2) |
| | FP-809 | 1.80 | 24.2 | (3.5) | 11.7 | (1.2) |
| | FP-809 | 3.75 | 31.7 | (0.9) | 18.5 | (0.7) |
| | FP-809 | 7.50 | 36.0 | (1.9) | 26.2 | (1.6) |
| MDA-MB-231 | | | | | | |
| | No MAb | 0 | 0 | (0) | 0 | (0) |
| | IgG1 | 10 | 0 | (0) | 0 | (0) |
| | FP-809 | 1.80 | 6.7 | (1.3) | 4.8 | (0.4) |
| | FP-809 | 3.75 | 13.9 | (0.5) | 9.5 | (0.1) |
| | FP-809 | 7.50 | 20.7 | (1.0) | 9.5 | (0.1) |

The inventors further examined whether pre-exposure of FP-809 to both NK cells and tumor cells may provide synergistic effect on cell lysis. In the first experiment, as schematically described in FIG. 7A, NK cells from were pre-exposed or incubated with FP-809 24 hours before being washed. Tumor cells were exposed to IgG1 control or FP-809 for 30 min before co-incubated with pre-exposed NK cells for the lysis assay at a 10:1 E:T ratio. As shown in FIGS. 7B-D, pre-exposure of FP-809 to NK cells or tumor cells could increase the cell lysis percentage. Importantly, pre-exposure of FP-809 to both NK cells and tumor cells could further increase the cell lysis percentage compared to pre-exposure of FP-809 to either NK cells or tumor cells (e.g., to 78% from 35% or 60% in H441 cells, etc.), indicating that pre-exposure of FP-809 to both NK cells and tumor cells render synergistic effect on cell lysis of tumor cell by NK cells.

Still further, the inventors examined whether the effect of FP-809 on NK cells to increase ADCC can be augmented by other anti-tumor reagent treatable to tumor cells. As shown in Table 7, protein expression level of PD-L1 and EGFR were measured in mean fluorescence intensity (MFI) by multicolor flow cytometry to determine the percent expression of such proteins. The inventors found that a majority of all three types of tumor cells (H441, CaSki, MDA-MB-231) shows PD-L1 and/or EGFR expression on the cell surface, indicating that targeting such proteins can effectively target the tumor cells.

TABLE 7

| Cell Line | PD-L1 (MFI) | EGFR (MFI) |
|---|---|---|
| H441 | 91.6 (1228) | 95.2 (1247) |
| CaSki | 78.7 (4599) | 98.4 (23318) |
| MDA-MB-231 | 66.5 (536) | 97.4 (1637) |

In the first experiment, as schematically described in FIG. 7E, NK cells from were pre-exposed or incubated with FP-809 24 hours before being washed. Tumor cells were exposed to IgG1 control or cetuximab for 30 min before NK cells were added. The inventors found that, as shown in FIGS. 7F-H, cetuximab treatment to the tumor cells before contacting FP-809 pre-exposed NK cells further increased cell lysis percentage compared to samples of pre-exposure of FP-809 to NK cell only or cetuximab treatment to the tumor cells only (e.g., to 56% from 35% (pre-exposure of FP-809 to NK cell only) or 20% (cetuximab treatment to the tumor cells only) in H441 cells, etc.), indicating that cetuximab treatment to the tumor cells can render synergistic effect on cell lysis of tumor cell by NK cells pre-exposed to FP-809.

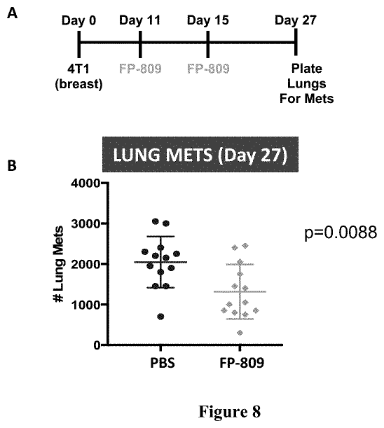

The inventors then further determined whether the FP-809, as a potent activator of ADCC, or cell lysis mediated by ADCC, can be effective to treat the tumor in vivo. As schematically shown in FIG. 8A, a healthy mouse was transplanted with 4T1 mammary carcinoma, which is a transplantable tumor cell line that is highly tumorigenic and invasive, and which can spontaneously metastasize from the primary tumor in the mammary gland to multiple distant sites. The 4T1-transplanted mouse was treated with FP-809 11 days and 15 days after the transplant, and in day 27 (12 days after the second FP-809 treatment), lung tissue of the 4T1-transplanted mouse was examined to see the number of metastasized tumor tissues. As shown in FIG. 8B, the inventors found that the number of metastasized tissue in the lung was significantly decreased (P=0.0088) with FP-809 treatment compared to untreated control.

### Use of Chimeric Molecule Complexes for Treating Tumor in a Patient

The inventors still further contemplate that the compounds and compositions that include chimeric molecule complexes can be administered to a patient having a tumor to increase the immune response against the tumor, especially ADCC against the tumor such that the tumor size can be decreased and/or the metastasis rate of the tumor can be reduced. As used herein, the term “administering” refers to both direct and indirect administration of the compounds and compositions contemplated herein, where direct administration is typically performed by a health care professional (e.g., physician, nurse, etc.), while indirect administration typically includes a step of providing or making the compounds and compositions available to the health care professional for direct administration.

It is contemplated that in some embodiments, chimeric molecules and complexes will be administered in formulations suitable for injection in therapeutically effective amounts. Therapeutically effective amounts can range from nano gram (ng) quantities to 100 mg, such as from 10 ng to 50 mg, from 10 μg to 10 mg. Co-solvents or detergents can be used to increase the storage stability and solubility of the formulation. Additionally, or alternatively, mixed-phase or two-phase liquid systems can be used. Most preferably, chimeric molecule complexes will be formulated in liquid ready to use. However, it is contemplated that the chimeric molecule complexes can also be formulated in a dried form for reconstitution via lyophilization or freeze drying.

In some embodiment, the chimeric molecule composition or formulation can be administered via systemic injection including subcutaneous, subdermal injection, or intravenous injection. In other embodiments, where the systemic injection may not be efficient (e.g., for brain tumors, etc.), it is contemplated that the formulation is administered via intratumoral injection.

With respect to dose and schedule of the formulation administration, it is contemplated that the dose and/or schedule may vary depending on depending on the type of chimeric molecule or molecule complex, type and prognosis of disease (e.g., tumor type, size, location), health status of the patient (e.g., including age, gender, etc.). While it may vary, the dose and schedule may be selected and regulated so that the formulation does not provide any significant toxic effect to the host normal cells, yet sufficient to induce ADCC against the tumor cells. Thus, in a preferred embodiment, an optimal or desired condition of administering the formulation can be determined based on a predetermined threshold. For example, the predetermined threshold may be a predetermined local or systemic concentration of cytokine (e.g., IFN-γ, IL-10, IL-13, etc.) released from activated NK cells. Therefore, administration conditions are typically adjusted to have the concentration of cytokine increased at least 20%, at least 30%, at least 50%, at least 60%, at least 70% at least locally or systemically. Alternatively, the dose and schedule of the formulation administration can be adjusted based on the periodic observation of the tumor size or tumor cells to have the tumor size decreased at least 10%, at least 20%, at least 30%, or at least 40%, within 14 days, within 28 days, or within 2 months, etc.

In some embodiments, especially where the chimeric molecule composition includes FP-809, the inventors contemplate that composition can be administered intratumorally in a lower dose to sensitive tumor cells to ADCC, then administered either intratumorally in a higher dose to activate CD4+, CD8+ T cells and/or NK cells to elicit immune response against the tumor cells. In such embodiments, the higher dose can be at least 50%, at least 2-fold, at least 3-fold, at least 5-fold higher than the lower dose.

Alternatively, the inventors also contemplate that immune competent cells (CD4+, CD8+ T cells, NK cells, NKT cells) can be pre-exposed to or treated with chimeric molecule composition (preferably including FP-809) ex vivo and then administered to the patient to treat tumor. In such embodiments, in order to reduce any allograft rejection, it is preferred that the immune competent cells are autologous cells such that they are isolated from the patient, or are the cells grown from the precursor cells of the patient. However, it is also contemplated that the immune competent cells are derived from any immortalized cell lines (typically irradiated prior to administration).

For example, with respect to NK cells, NK cells can be readily identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify and/or isolate NK cells, using methods well known in the art. Of course, it should be noted that suitable host cells, and particularly NK cells are either obtained from the patient diagnosed with the tumor, or are obtained from an already established cell line as further detailed below.

With respect to NKT cells, NKT cells represent a heterogeneous cell population that can be grouped into three categories based on presence of several molecular markers (e.g., Vα24, etc.) and/or their reactivity to a ligand (e.g., CD1d-restricted, reactivity to α-galactosylceramide (α-GalCer), etc.). In one embodiment, isolation of human type I NKT cells, which typically express Vα24-Jα18 type T cell receptor, can be performed using an antibody against Vα24 or an antibody against Vα24-Jα18. In other embodiments, isolation of human type I and type II NKT cells, which are typically CD1d-restricted cells, can be performed using a portion of CD1d molecule (preferably the portion that are responsible for a high affinity to NKT T cell receptor), a portion of CD1d molecule coupled with a lipid antigen (e.g., any lipid antigens that are generated from a foreign organism, nutritional substances, or self-lipids generated from the patient that can bind to CD1d, etc.), or a portion of CD1d molecule coupled with a peptide (e.g., p99, etc.). Once isolated, the population of isolated and enriched NKT cells can be further increased via ex vivo expansion of the NKT cells. The ex vivo expansion of NKT cells can be performed in any suitable method with any suitable materials that can expand NKT cells at least 10 times, preferably at least 100 times in 7-21 days. For example, isolated and enriched NKT cells can be placed in a cell culture media (e.g., AIMV® medium, RPMI1640®, etc.) that includes one or more activating conditions. The activating conditions may include addition of any molecules that can stimulate NKT growth, induce cell division of NKT, and/or stimulate cytokine release from NKT that can further expand NKT cells. Thus the activating molecules include one or more cytokines (e.g., IL-2, IL-5, IL-7, IL-8, IL-12, IL-12, IL-15, IL-18, and IL-21, preferably human recombinant IL-2, IL-5, IL-7, IL-8, IL-12, IL-12, IL-15, IL-18, and IL-21, etc.) in any desirable concentration (e.g., at least 10 U/ml, at least 50 U/ml, at least 100 U/ml), T cell receptor antibodies (e.g., anti-CD2, anti-CD3, anti-CD28, α-TCR-Vα24+ antibodies, preferably immobilized on beads, etc.), a glycolipid (e.g., α-GlcCer, β-ManCer, GD3, etc.), a glycolipid coupled with CD1 (e.g., CD1d, etc.), etc.

With respect to T cells, T cells can be CD4+ and/or CD8+ T cells that may be naive to the patient or allogeneic are contemplated. Compared to NK cells or NKT cells, the frequencies of T cells within the lymphocytes is generally high (e.g., 25-60% for CD4+ T cells among peripheral blood mononuclear cells (PBMC) and 5-30% for CD8+ T cells among PBMC)), and any suitable method to isolate T cells (CD3+) or subtypes of T cells (CD4+ or CD8+) using a marker (e.g., CD3, CD4, CD8, etc.) are contemplated including Fluorescence-activated cell sorting (FACS), and any pull-down assays.

However, it is also contemplated that the NK, NKT, T cells may also be heterologous NK, NKT, T cells. For example, preferred NK cells may include immortalized NK cells (typically irradiated prior to administration), and such immortalized NK cells include NK92 cells that may be genetically engineered to achieve one or more specific purpose. For example, NK cell is a NK92 cell that has a recombinant high affinity variant of CD16 (e.g., V158 variant). In addition, it is also preferred that the NK92 cell is further genetically modified to express IL-2 in the endoplasmic reticulum such that the cytotoxicity of NK cell remains active under hypoxic conditions (e.g., tumor microenvironment). One of the preferred types of NK cells includes commercially available haNK cells from NantKwest (9920 Jefferson Blvd. Culver City, Calif. 90232).

Optionally, the isolated immune competent cells from the patient can be further expanded ex vivo. The ex vivo expansion of NK cells or NKT cells can be performed in any suitable method with any suitable materials that can expand NK cells or NKT cells at least 10 times, preferably at least 100 times in 7-21 days. For example, NK cells or NKT cells can be placed in a cell culture media (e.g., AIMV® medium, RPMI1640®, etc.) that includes one or more activating conditions. The activating conditions may include addition of any molecules that can stimulate NK or NKT growth, induce cell division of NK or NKT, and/or stimulate cytokine release from NK or NKT that can further expand NK or NKT cells. It is contemplated that the activating conditions may vary depending on the timing of the ex vivo expansion and activation. For example, NK cell expansion can be performed using various activating molecules added in the culture media including cytokines (e.g., IL-2, IL-15, etc.), monoclonal antibodies (e.g., murine monoclonal antibody against CD3 (OKT3™), etc.), or using cell-to-cell interaction with activating cells (e.g., K562 cells, a cell line derived from a patient with myeloid blast crisis of chronic myelogenous leukemia and bearing the BCR-ABL1 translocation, etc.)

With respect to NKT cells, ex vivo expansion and activation of NKT cells can be performed using the activator of endogenous NKT T cell receptor or antibodies against the components of the endogenous NKT T cell receptor, before the endogenous NKT T cells are removed by knock-in of recombinant nucleic acid. However, after the endogenous NKT T cells are removed by knock-in of recombinant nucleic acid, it is contemplated that the activator of endogenous NKT T cell receptor or antibodies against the components of the endogenous NKT T cell receptor may not be used for effective ex vivo expansion and activation.

Thus, the activating molecules may include T cell receptor antibodies (e.g., anti-CD2, anti-CD3, anti-CD28, α-TCR-Vα24+ antibodies, preferably immobilized on beads, etc.), a glycolipid (e.g., α-GlcCer, β-ManCer, GD3, etc.), a glycolipid coupled with CD1 (e.g., CD1d, etc.) if the ex vivo expansion and activation is performed before the recombinant nucleic acid is introduced into the NKT cells. After the recombinant nucleic acid is introduced into the NKT cells, the activating molecules may include one or more cytokines (e.g., IL-2, IL-5, IL-7, IL-8, IL-12, IL-12, IL-15, IL-18, and IL-21, preferably human recombinant IL-2, IL-5, IL-7, IL-8, IL-12, IL-12, IL-15, IL-18, and IL-21, etc.) in any desirable concentration (e.g., at least 10 U/ml, at least 50 U/ml, at least 100 U/ml), etc. In some embodiments, the activation conditions may include culturing the isolated and enriched NKT cells with autologous or allogeneic peripheral blood mononuclear cells (PBMC) feeder cells.

In addition, the inventors also contemplate that the ex vivo expansion of the NKT cells can be directed to expansion of specific types of NKT cells by treating the NKT cells with different types of glycolipids (e.g., α-GlcCer, β-ManCer, GD3, etc.) that may trigger NKT cells having different profiles of cytokine release. For example, NKT cells can be treated with extracellular α-GlcCer ex vivo to induce expanded NKT cells to a particular type: IFN-γ producing NKT cells. For other example, NKT cells can be treated with extracellular β-ManCer ex vivo to induce expanded NKT cells to another particular type: NKT cells with TNF-α, iNOS-dependent antitumor activity.

With respect to the activating conditions, it is contemplated that the dose and schedule of providing activating conditions may vary depending on the initial number of NK cells or NKT cells and the condition of NK cells or NKT cells. In some embodiments, a single dose of cytokine (e.g., 100 U/ml) can be employed for at least 3 days, at least 5 days, at least 7 days, at least 14 days, at least 21 days. In other embodiments, the dose of cytokine may be increased or decreased during the expansion period (e.g., 200 U/ml for first 3 days and 100 U/ml for next 14 days, or 100 U/ml for first 3 days and 200 U/ml for next 14 days, etc.). Also it is contemplated that different types of cytokines can be used in combination or separately during the ex vivo expansion (e.g., IL-15 for first 3 days and IL-18 for next 3 days, or combination of IL-15 and IL-18 for 14 days, etc.).

Such isolated and optionally expanded/activated immune competent cell can then be contacted with chimeric molecule composition (e.g., FP-809) ex vivo. The dose and schedule of treating the immune competent cell with the chimeric molecule composition ex vivo may vary depending on the type of chimeric molecule composition and the type of immune competent cells. For example, where the chimeric molecule is FP-809, and the immune competent cells are NK cells and/or T cells (CD4+, CD8+), FP-809 can be treated to the NK cells and/or T cells (CD4+, CD8+) at a concentration in any physiological range of 1 ng/ml-100 ng/ml, 3 ng/ml-70 ng/ml, 5 ng/ml-60 ng/ml, for at least 6 hours, at least 12 hours, at least 24 hours, at least 2 days before administering the immune competent cell to the patient. In some embodiments, the effectiveness of ex vivo FP-809 treatment can be determined before administering the cells to the patient by further isolating a portion of the a pre-exposed or treated immune competent cells to determine gene expression and/or protein expression level or by determining the secreted cytokine level in the cell culture medium. For example, FP-809 can be treated to the NK cells at a concentration and a duration such that the amount of IFN-γ secreted by NK cells increases at least 50 folds, at least 100 folds, at least 200 folds, or even 500 folds compared to untreated NK cells.

It is contemplated that the ex vivo, pre-exposed (treated) immune competent cells can be formulated in any pharmaceutically acceptable carrier (e.g., as a sterile injectable composition) with a cell titer of at least 1×10³ cells/ml, preferably at least 1×10⁵ cells/ml, more preferably at least 1×10⁶ cells/ml, and at least 1 ml, preferably at least 5 ml, more preferably and at least 20 ml per dosage unit. However, alternative formulations are also deemed suitable for use herein, and all known routes and modes of administration are contemplated herein. In some embodiments, the cell composition can comprise homogeneous pre-exposed (treated) immune competent cells (e.g., pre-exposed NK cells). In other embodiments, the cell composition may comprise heterogeneous pre-exposed (treated) immune competent cells (e.g., pre-exposed NK cells and CD4+ T cells). It is also contemplated that the cell composition can comprise mixed groups of immune competent cells that are pre-exposed to chimeric molecule composition in different conditions. For example, the cell composition may include two groups of NK cells mixed together that one group of NK cells are pre-exposed to FP-809 at a concentration of 5 ng/ml for 24 hours and another group of NK cells are pre-exposed to FP-809 for 30 ng/ml for 18 hours, and the ratio between two group of cells can be at least 1:1, at least 2:1, at least 3:1, at least 5:1, or at least 1:2, at least 1:3, or at least 1:5.

Co-Administration of Anti-Cancer Drug with Pre-Exposed Immune Competent Cells

Preferably, administration of pre-exposed immune competent cells to the cancer patient can be accompanied with intratumoral or systemic administration of one or more of chimeric molecule composition, an anti-cancer drug, or another cancer therapy. For example, an intratumoral injection of a formulation including FP-809 can be accompanied with administration of pre-exposed immune competent cells in any manner to elicit a synergistic effect of increasing ADCC against the tumor cell. In these examples, FP-809 formulation can be administered at least 30 min, at least 1 hour, at least 2 hours, at least 6 hours before administration of pre-exposed immune competent cells to the patient such that the tumor cells can be pre-sensitized to ADCC by pre-exposed immune competent cells to so increase the tumor cell lysis. Yet, it is also contemplated that the FP-809 formulation can be mixed with the pre-exposed immune competent cells to be administered intratumorally.

Additionally or alternatively, intratumoral or systemic injection of anti-cancer drug can be accompanied with administration of pre-exposed immune competent cells in any manner to elicit a synergistic effect of increasing ADCC against the tumor cell. Any suitable anti-cancer drug, including peptide-based, antibody-based, nucleic acid-based, and non-peptide based molecules are contemplated that can be coupled to the immune competent cell without incurring substantial interference to the cell's expected activity (e.g., recognition of an antigen, etc.) and without substantial loss of cytotoxicity in the extracellular environment (and potentially mildly acidic environment such as tumor microenvironment). Thus, especially preferred anti-cancer drugs may include an immune-stimulating cytokine, an immune-stimulating chemokine, a radiosensitizing drug, or a chemotherapeutic drug. For example, the anti-cancer drugs may include one or more immune-stimulatory molecules (e.g., CD30L, CD40L, ICOS-L, OX40L, 4-1BBL, GITR-L, etc.), immune stimulatory cytokines (e.g., IL-2, IL-12, IL-15, IL-15 super agonist (ALT803), IL-21, IPS1, and LMP, etc.), and/or checkpoint inhibitors (e.g., antibodies or binding molecules to CTLA-4 (especially for CD8⁺ cells), PD-1 (especially for CD4⁺ cells), TIM1 receptor, 2B4, and CD160, etc.).

In other examples, the anti-cancer drugs can be one or more of MDSC inhibitors that includes MDSC recruitment inhibitor, MDSC expansion inhibitor, MDSC differentiation inhibitor, and/or MDSC activity inhibitor. MDSC recruitment inhibitor may include one or more antagonists of one or more colony-stimulating factor 1 receptor (CSF-R), granulocyte colony-stimulating factor (G-CSF), C—C motif chemokine ligand 2 (CCL2), or C—X—C chemokine receptor type 4 (CXCR4). The antagonist may include small molecule inhibitors, antibodies or fragments thereof that bind to the target molecule, single-chain variable fragment (scFv) molecule binding to the target molecule, or any other suitable binding molecules. For example, the antagonist of CSF-R may include a small molecule inhibitor (e.g., Pexidartinib, etc.) or one or more monoclonal antibodies against CSF-R (e.g., Emactuzumab, AMG820, imc-CS4, MCS110, etc.) Alternatively or additionally, expansion of the MDSCs in the tumor may be inhibited by administering gemcitabine, amino bisphosphonates, sunitinib, or celecoxib, and differentiation of MDSCs in the tumor may be inhibited by taxanes, curcumin, or Vitamin D3. In addition, MDSC activity in the tumor may be inhibited by administration of amiloride, CpG, COX2 inhibitors, PDE-5 inhibitors, or PGE2 inhibitors.

Additionally, or alternatively, the anti-cancer drug may be a CXCR1 inhibitor and/or a CXCR2 inhibitor. There are various such inhibitors known in the art, and appropriate inhibitors various 2-amino-3-heteroaryl-quinoxalines (see e.g., Bioorg Med Chem. 2003 Aug. 15;11(17):3777-90), 6-Chloro-3-[[[(2,3-dichlorophenyl)amino]carbonyl]amino]-2-hydroxybenzenesulfonamide (SB332235), or N-(2-Bromophenyl)-N′-(7-cyano-1H-benzotriazol-4-yl)urea (SB265610). If inhibitors with higher specificity are desired, SCH-527123 and SCH-479833 may be employed that will selectively inhibit CXCR2 and CXCR1, respectively (see e.g., Clin Cancer Res. 2009 Apr. 1; 15(7):2380-6). Of course, it should be appreciated that the CXCR1/2 pathway activity may also be inhibited by one or more agents that interfere with the elements of the signaling chain. In still another example, the activation of the IL-8 receptor, including CXCR1/2, can be inhibited using reparixin (also known as repertaxin, see e.g., Biol Pharm Bull. 2011; 34(1):120-7), or the IL-8-mediated signaling cascade through CXCR1/2 can be inhibited by blocking one or more elements in the signaling pathways. Thus, inhibitors can also target CXCR1 and 2 signaling pathways by targeting PI3kinase, pAkt, or mTOR for CXCR1 signaling inhibition, and/or RhoGTPase, RacGTPase, and Ras, Raf, Mek, or pErk for CXCR2 signaling inhibition. Since IL-8 signaling also at least indirectly affects MDSCs, it is expected that at least some of the above agents will reduce activity or recruitment of MDSC to the tumor environment.

Still further, the anti-cancer drug may be a reagent that inhibit EMT of the tumor cell or reverse the EMT process of the tumor cell, or even promote mesenchymal to epithelial transition (MET) of the tumor cell. For example, during the EMT process, TGF-β induces isoform switching of FGF Receptor 2 (e.g., from isotype IIIb to IIIc), and it is contemplated that inhibiting TGF-β activity in the tumor cells (e.g., using dominant negative form of TGF-β RII, monoclonal antibodies against TGF-beta 1 and beta 2, including lerdelimumab and metelimumab, etc.) may reduce or prohibit the isoform switching of FGF Receptor 2 to so prevent EMT of the tumor cell. In yet another example, MET may be induced in vitro by administering 8-bromo-cAMP, Taxol, or Adenosine 3′,5′-cyclic Monophosphate, N6-Benzoyl-Sodium Salt, which activate protein kinase A (PKA). MET of the tumor cell can be also induced by administering a recombinant virus encoding recombinant E-Cadherin or regulatory RNA inhibiting N-Cadherin expression to stimulate of E-Cadherin overexpression and reduce N-Cadherin expression. Further, MET of the tumor cell can be also induced by EGFR inhibition and/or down-regulation of Snail, Slug, Zeb-1, Zeb-2, and/or N-cadherin (e.g., using siRNA, miRNA, shRNA, or other regulatory small molecule reducing the post-transcriptional expression, etc.).

It is also contemplated that the anti-cancer drug may include other inhibitory reagents to immune suppressive cells may be administered concurrently with the binding molecule and/or cytotoxic immune cells or before administering the binding molecule and/or cytotoxic immune cells. Especially contemplated reagents include RP-182 to inhibit or kill M2 macrophages, gemcitabine, cis-platinum, and/or cyclophosphamide to reduce or inhibit regulatory T cells (Tregs).

For still other example, preferred and suitable anti-cancer drugs may include site-specific radioisotope treatment using therapeutic alpha and/or beta emitters. Suitable alpha emitters include actinium-225 (²²⁵Ac, 10 d), astatine-211 (²¹¹At, 7.2 h), bismuth-212 (²¹²Bi, 1 h), bismuth-213 (²¹³Bi, 45.6 min), radium-223 (²²³Ra, 11.4 d), actinium-225 (²²⁵Ac, 10.0 d) and thorium-227 (²²⁷Th, 18.7 d), while suitable beta emitters include tungsten-188 (¹⁸⁸W, 69.4 d), Yttrium-90 (⁹⁰Y, 64.1 hours), copper-67 (⁶⁷Cu, 61.8 h), copper-64 (⁶⁴Cu, 12.7 h), rhenium-186 (¹⁸⁶Re, 70 d) strontium-90 (⁹⁰5 r, 28.8 y), and Lutetium-177 (¹⁷⁷Lu, 160.4 d). In some embodiments, an auger-emitters can be used in limited circumstances. Suitable auger emitters include gallium-67 (⁶⁷Ga), iodine-123 (¹²³I) and iodine-125 (¹²⁵I). The alpha and/or beta emitters (and sometimes auger emitters) may be coupled to a peptide carrier (e.g., tumor-specific peptide, etc.) or loaded on a biocompatible nanoparticle (e.g., colloidal gold particle, quantum dot, photosensitive gold nanoparticles, magnetic nanoparticles, as well as polymer based polymeric nanoparticles and nanoscale liposomes, etc.).

While the dose and the schedule of anti-cancer drug administration may vary depending on the type of anti-cancer drug, it is contemplated that In these examples, the anti-cancer drug formulation can be administered at least 30 min, at least 1 hour, at least 2 hours, at least 6 hours before administration of pre-exposed immune competent cells to the patient such that the tumor cells can be pre-sensitized to ADCC by pre-exposed immune competent cells to so increase the tumor cell lysis. Yet, it is also contemplated that the anti-cancer drug formulation can be mixed with the pre-exposed immune competent cells to be administered intratumorally.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. A chimeric molecule comprising an Fc portion of an antibody and an immunomodulatory portion,
wherein the immunomodulatory portion comprises at least two immune effectors,
wherein the Fc portion comprises at least a portion of an IgG antibody,
wherein the immune effector comprises at least a portion of PD-L1, a PD-L1 binder, PD-1, a PD-1 binder, PD-L2, a PD-L2 binder, 4-1BBL, OX40L, and/or GITRL.

2.-3. (canceled)

4. The chimeric molecule of claim 1, wherein the at least two immune effectors are in a concatemeric arrangement.

5. The chimeric molecule of claim 1, wherein the at least two immune effectors are at two distinct portions of the chimeric molecule.

6. The chimeric molecule of claim 1, further comprising a cytokine receptor and a cytokine or cytokine analog bound to the cytokine receptor.

7. (canceled)

8. The chimeric molecule of claim 1, further comprising an affinity portion covalently bound to at least one of the cytokine receptor, the cytokine, and the cytokine analog.

9. (canceled)

10. The chimeric molecule of claim 1, further comprising at least one of a cytokine, a cytokine analog, and an affinity portion.

11. The chimeric molecule of claim 1, wherein the chimeric molecule is a first chimeric molecule and further comprises a second chimeric molecule comprising features of the first chimeric molecule, wherein the first and second chimeric molecules are bound together at respective Fc portions via a disulfide bond.

12.-20. (canceled)

21. A recombinant immunoglobulin protein complex, comprising:
an Fc domain having first and second Fc portions coupled with respective first and second cytokine binding domains having respective first and second target recognition domains;
wherein the first and second cytokine binding domains are coupled with a first and second cytokines; and
wherein at least one of the first and second target recognition domains is configured to bind PD-L1.

22. The protein complex of claim 21, wherein the first and second Fc portions form a dimer.

23. The protein complex of claim 21, wherein at least one of the first and second cytokine binding domain is IL-15Ra.

24. (canceled)

25. The protein complex of claim 21, wherein at least one of the first and second cytokines is IL-15.

26. The protein complex of claim 21, wherein the at least one of the first and second target recognition domains is selected from a group consisting of: a PD-L1 antibody, Fab fragment of a PD-L1 antibody, scFv fragment binding to PD-L1.

27. The protein complex of claim 21, wherein the protein complex is coupled with a carrier protein via the Fc domain, and wherein the carrier protein is selected from the following: protein A, protein G, protein Z, albumin, or refolded albumin.

28. (canceled)

29. A method of modulating gene expression in an immune competent cell, comprising:
providing a recombinant immunoglobulin protein complex, comprising:
an Fc domain having first and second Fc portions coupled with respective first and second cytokine binding domains having respective first and second target recognition domains;
wherein the first and second cytokine binding domains are coupled with a first and second cytokines;
wherein at least one of the first and second target recognition domains is configured to bind PD-L1; and
contacting the recombinant immunoglobulin protein complex to the immune competent cell in a dose and schedule effective to modulate gene expression.

30. The method of claim 29, wherein the first and second Fc portions form a dimer.

31. The method of claim 29, wherein at least one of the first and second cytokine binding domain is IL-15Ra, and wherein at least one of the first and second cytokines is IL-15.

32.-33. (canceled)

34. The method of claim 29, wherein the at least one of the first and second target recognition domains is selected from a group consisting of: a PD-L1 antibody, Fab fragment of a PD-L1 antibody, scFv fragment binding to PD-L1.

35. The method of claim 29, wherein the protein complex is coupled with a carrier protein via the Fc domain, wherein the carrier protein is selected from the following: protein A, protein G, protein Z, albumin, refolded albumin.

36. (canceled)

37. The method of claim 14, wherein the immune competent cell is at least one of CD4+ T cell, CD8+ T cell, and NK cell.

38. The method of claim 14, wherein the gene expression is modulated to increase mRNA expression of at least two genes for at least two folds.

39.-81. (canceled)
